(19) United States
(12) Patent Application Publication
(10) Pub. No.: US 2003/0228264 A1
(43) Pub. Date: Dec. 11, 2003

(54) Dissolvable teeth whitening apparatus

(76) Inventor: Salvatore F. Perna, Ladera Ranch, CA (US)

Correspondence Address: KOESTNER BERTANI LLP, 18662 MACARTHUR BLVD, SUITE 400, IRVINE, CA, 92612, US

(21) Appl. No.: 10/456,384

(22) Filed: Jun. 6, 2003

Related U.S. Application Data
(60) Provisional application No. 60/387,985, filed on Jun. 6, 2002.

(51) Int. Cl. A61K 9/68; A61K 7/20

(52) U.S. Cl. 424/53

(57) ABSTRACT

A teeth whitening system comprises a dissolvable matrix that supports a whitening material. The combination of dissolvable matrix and whitening material is attached to one or more teeth so that the whitening material is exposed to the one or more teeth over time as the dissolvable matrix slowly dissolves. In some examples, the combination is applied to a tooth with the whitening material adjacent to the tooth. In some examples, the whitening material is contained within one or more vesicles, pockets, or reservoirs within the dissolvable matrix and is placed in contact with the teeth by crushing or adhering the dissolvable matrix to the teeth. In still other examples, the whitening material is mixed with the dissolvable matrix. In some examples, the whitener is composed of precursor materials that are separated until usage, for example by distributing the different precursors in separate vesicles until pressed against one or more teeth for usage. Teeth whitening units can be any suitable size and shape for application to one or more teeth, for example by pressing or crushing the unit against a tooth.

### BACKGROUND OF THE INVENTION

White teeth are commonly acknowledged to be more attractive than discolored teeth. Teeth lose a white appearance for many reasons including staining from certain foods such as cherries, blueberries, blackberries, and dark green vegetables. Consumption of colored beverages including sodas, coffee, tea, wine, and many others also causes staining. Activities such as tobacco usage, exposure to antibiotics, genetic traits, and aging also result in tooth discoloration. Thus, the presence of chromogenic agents in food, beverages, tobacco, and salivary fluid, as well as internal exposure through blood, amalgam restoratives, and antibiotics such as tetracycline, nearly invariably cause teeth to become discolored.

Multiple portions of the teeth can become stained including enamel, dentin, and the acquired pellicle. Tooth enamel is predominantly constructed of inorganic material, mostly hydroxyapatite crystals, and contains approximately 5% organic material as primarily collagen. Dentin is about 20% protein and includes collagen and inorganic material, mostly hydroxyapatite crystals. Acquired pellicle is a protein layer on the surface of tooth enamel that is easily whitened by intensive tooth cleaning.

Staining of the multiple portions of teeth results from different processes, internal and external. Extrinsic staining of the acquired pellicle results from compounds including tannins and other polythenolic compounds that are trapped and tightly bound to the proteinaceous layer on a tooth surface, and is generally removed by mechanical tooth cleaning methods such as brushing. Intrinsic staining involves penetration of staining materials into the enamel and the dentin, or arises from sources internal to the teeth, and is not removed by mechanical or chemical external tooth cleaning. Intrinsic tooth stains are more difficult to remove than external stains.

Two categories of tooth bleaching systems exist. First, compositions such as toothpastes or gels that are applied to the teeth and mechanically agitated at the stained tooth surface to remove stain by abrasive erosion of the stained acquired pellicle. Second, gels, pastes, or liquids that bleach teeth by a chemical process while in contact with the stained tooth surface for a specified period, and are then removed. Either category may include an auxiliary chemical process, either oxidative or enzymatic, that supplements the mechanical process. Home bleaching systems have become available that allow individuals to whiten discolored or stained teeth. Typically home bleaching systems use weak oxidizing agents such as 6 to 15 percent carbamide peroxide or other peroxy compound.

In one type of system, a user delivers the oxidizing agents generally using a rigid dental appliance or tray that is custom-fabricated by a dentist or dental laboratory. The user is normally expected to wear the rigid dental appliance for up to 120 minutes per day, usually over a several week period. The available bleaching agents are either viscous liquids or gels that are commercially available and packaged in separate dispensing containers such as bottles, syringes, and tubes. The bleaching agent is self-administered by the patient by dispensing to the rigid custom dental appliance. The home bleaching technique requires dental office visits for acquisition of dental arch impressions and fabrication of a rigid custom dental appliance.

Many problems and inconveniences occur while obtaining rigid custom-fabricated dental appliances including the time and expense of making dental impressions and dental laboratory work, office visits, and possible reshaping of poorly fitted appliances. Other difficulties include occlusion and retention of bleaching agent in the appliance, inconvenience in cleaning and maintenance of the foam-lined dental appliance due to high surface area and pore volume, irritation to the user's lip when the appliance contacts tissue. Other whitening systems do not require construction of a custom appliance, but instead use a foam tray that is not fitted for application of the bleaching compounds. These systems have other drawbacks. The porous foam tray is bulky, lacks adequate structural rigidity to fit securely over the dental arches, and causes excessive salivation. Also, the foam appliances fail to direct and confine the application of home bleaching agents to patient teeth surfaces, reducing safety and utility of the system. Foam surfaces become saturated with bleaching agent and are open and exposed to the oral cavity, allowing elution of bleaching agent to enter the oral cavity and be ingested. Accordingly, the foam devices are inconvenient, irritating and uncomfortable.

Additional difficulties are common with whitening systems that employ a dental appliance. Users often fail to properly maintain, clean, and store the dental appliance to perform adequately. Users also may disperse excessive amounts of bleaching or other medicinal agent into the dental appliance and ingested, possibly causing discomfort or hypersensitive reactions including gingival irritation, burning, edema, nausea and other allergic reactions.

Other whitening systems do not utilize a dental appliance but instead use solid whitening strips that contain a bleaching agent. The strips are attached to the teeth and held in place for a specified time. The user removes the strips from the teeth when finished. A difficulty with whitening strips is that the necessary prolonged insertion of the strip on the teeth is often irritating and painful to soft tissues in the mouth. In addition, the bleaching agent is often inadequately distributed on the teeth with the sides of the teeth and depressed areas between the teeth having inadequate exposure to the whitening compounds.

What is needed is a teeth-whitening system that avoids the discomfort, irritation, and inconvenience of existing systems.

### SUMMARY OF THE INVENTION

A teeth whitener comprises a dissolvable matrix that is capable adhering to a dental surface and dissolving in the presence of oral fluids over a time period of minutes or hours, and a whitening material supported by the dissolvable matrix.

A teeth whitening system comprises a dissolvable matrix that supports a whitening material. The combination of dissolvable matrix and whitening material is attached to one or more teeth so that the whitening material is exposed to the one or more teeth over time as the dissolvable matrix slowly dissolves. In some examples, the combination is applied to a tooth with the whitening material adjacent to the tooth. In some examples, the whitening material is contained within one or more vesicles, pockets, or reservoirs within the dissolvable matrix, and is placed in contact with the teeth by crushing or adhering the dissolvable matrix to the teeth. In still other examples, the whitening material is mixed with the dissolvable matrix. In some examples, the whitener is composed of precursor materials that are separated until usage, for example by distributing the different precursors in separate vesicles until pressed against one or more teeth for usage. Teeth whitening units can be any suitable size and shape for application to one or more teeth, for example by pressing or crushing the unit against a tooth.

### BRIEF DESCRIPTION OF THE DRAWINGS

The features of the described embodiments believed to be novel are specifically set forth in the appended claims. However, embodiments of the invention relating to both structure and method of operation, may best be understood by referring to the following description and accompanying drawings.

FIG. 1 is a pictorial drawing showing an example of a tooth whitening apparatus.

FIG. 2 is a schematic pictorial drawing showing an example of a tooth whitening apparatus that includes two precursor materials that are combined to form a whitening material.

FIG. 3 is a pictorial drawing showing an example of a teeth whitening apparatus that includes a whitening material mixed into a dissolvable matrix.

FIG. 4 is a pictorial drawing that illustrates an example of a teeth whitening system including precursor materials distributed in vesicles or reservoirs throughout a dissolvable matrix.

FIG. 5 is a schematic pictorial drawing that shows several examples of suitable shapes for the teeth whitening apparatus.

FIG. 6 is a pictorial drawing showing an example of a suitable packaging configuration for the teeth whitening apparatus.

FIG. 7 is a pictorial drawing showing a second example of a suitable packaging configuration for the teeth whitening apparatus.

### DESCRIPTION OF THE EMBODIMENT(S)

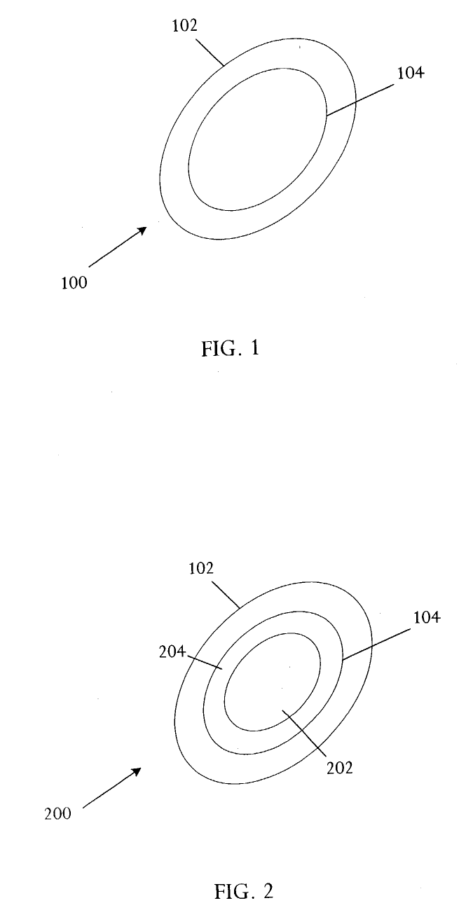

Referring to FIG. 1, a pictorial diagram illustrates an example of a teeth whitening apparatus 100. The teeth whitening apparatus 100 includes a dissolvable substrate 102 and a whitening material 104. The dissolvable substrate 102 may be in the form of a dissolvable matrix such as a gelatinous or protein material.

The dissolvable substrate 102 is composed of a material that is dissolvable within the mouth of a user and functions as a structural enclosure for the whitening material 104. The dissolvable substrate 102 has suitable flexibility for conforming to the shape of a tooth or several teeth, is easily cut or shaped for application to one or more teeth, and is capable of adhering to the enamel surface of a tooth. The dissolvable substrate 102 has a suitable strength for holding the whitening material 104 for a reasonable time of application that is typically in the range of minutes although may extend from a few seconds to a few hours.

The dissolvable substrate 102 is a material, for example a dissolvable matrix that dissolves on the teeth over time. In some examples, typical gelatinous materials may dissolve for a time period from a few minutes to over an hour, depending on the material and the in-mouth environment of a user. The dissolvable substrate 102 encapsulates the whitening material 104 and holds the whitening material 104 against one or more of the user's teeth until the substrate dissolves.

The dissolvable substrate 102 may be a gelatinous encapsulated matrix with a reservoir into which the whitening material 104 such as a bleaching agent may be filled. The dissolvable substrate 102 operates not only to sustain the application of the whitening material 104 to the teeth by virtue of a slow dissolving action, but also functions as an adhesive for holding the whitening material 104 to the teeth.

The dissolvable substrate 102 may take any suitable form for example as a solid form, a perforated form, or any combination. The dissolvable substrate 102 may be either pliable or rigid. The dissolvable substrate 102 may include any suitable therapeutic materials or compounds, such as fluoride or a sodium nitrate material. The dissolvable substrate 102 may be any color or translucent. The dissolvable substrate 102 may be flavored or unflavored.

In a particular example, the dissolvable substrate 102 may be a gelatin material having a structural rigidity, for example from 150 blume to 200 blume, or any combination, to yield a suitable rate of dissolution and structural integrity for holding the whitening material 104.

The dissolvable substrate 102 can be composed of a variety of materials. In one example, the dissolvable substrate 102 is a gelatin tablet such as is typically used as a vitamin E supplement. Gelatin tablets may have a variety of compositions. In one example, a suitable gelatin tablet is a mixture of vegetable oil, gelatin, glycerin, and water. Suitable vegetable oils include but are not limited to soybean oil, safflower oil, canola oil, corn oil, flax seed oil, and the like.

In another example, the dissolvable substrate 102 is composed of a cellulose and acrylic polymer. In a specific example, a suitable dissolvable substrate 102 is a freeze-dried composition of 0.5% sodium carboxymethylcellulose and 0.1% carbopol that is compounded using purified water and sodium hydroxide as a neutralizer. The freeze-dried dissolvable substrate 102 is formed into a solid sheet that is flexible and adherent to teeth.

In another example, the dissolvable substrate 102 is a dissolvable matrix composed of materials such as carbohydrate, fat, protein, wax (natural or synthetic), dissolvable resin, hydrogel, or other suitable dissolvable material. The dissolvable substrate 102 may be flavored or unflavored and may include or omit various other substances having clinical effect.

The dissolvable substrate 102 may contain any suitable compounds or elements such as fluoride or sodium nitrates, for example to reduce tooth sensitivity.

In some embodiments, the dissolvable substrate 102 may completely encapsulate the whitening material 104. The user presses the teeth whitening apparatus 100 against one or more teeth to expose the teeth to the whitening material 104 and begin the whitening action.

The dissolvable substrate 102 may be formed to completely encapsulate the whitening material 104 or may simply function as a reservoir for holding the whitening material 104. For example, in one configuration, the dissolvable substrate 102 may be in the form of a gelatin capsule that completely contains the whitening material 104 in one or more inner pockets. The user may press the capsule against a tooth causing the whitening material 104 to contact the tooth. In some configurations, the capsule may be inserted over a tooth using the tooth to cut through the dissolvable substrate 102 and expose the tooth to the whitening material 104.

In another example, the dissolvable substrate 102 in the form of a gelatin capsule may have a single reservoir that does not completely enclose the whitening material 104. For example, the dissolvable substrate 102 may be a gelatin cup that may be mounted on a mounting strip so that the whitening material 104 is contained between the gelatin cup and the mounting strip. A user typically removes a gelatin cup from the mounting strip and presses the cup against a tooth so that the whitening material 104 contacts the tooth and the dissolvable substrate 102 adheres to the tooth.

In other embodiments, the whitening material 104 may be coat one side of the dissolvable substrate 102. The user may obtain best results by applying the teeth whitening apparatus 100 to one or more teeth with the whitening material 104 adjacent to the teeth.

The user places the teeth whitening apparatus 100 onto one or more teeth for an extended time so that the whitening material 104 contacts the teeth while the dissolvable substrate 102 dissolves.

In some configurations, a layer of whitening material 104 or multiple layers of whitening material 104 may be interspersed between layers of a dissolvable composition. A variety of dissolvable matrix materials may be compressed, poured, dried, or otherwise formed into a solid but flexible form, virtually any desired type of mold can be used to determine the shape and size of the dissolvable substrate 102. In some examples, multiple pieces of the dissolvable substrate 102 and the whitening material 104 can be assembled in any form using a dissolvable bonding agent such as a confectioner's glue.

The dissolution rate of the matrix may be controlled by including a hydrophobic agent such as calcium stearate to slow dissolution or lactose to enhance dissolution. Alternatively, the matrix material, for example gelatin, fat, protein, wax, may be selected according to the dissolution rate. Also the extent of mechanical compression of the mixture material may be controlled to adjust dissolution rate.

In some examples, the dissolvable matrix is formed by thoroughly mixing the desired constituents into a solid dosage-form. In other cases the constituents are wetted to form a slurry, dried, and then compressed so that compressed powdered matrix is held together physically rather than chemically. The extent of compression may be selected to attain a desired dissolution rate. In other examples, constituents may be formed into a dosage-form by dehydration, freeze-drying or lyophilization, molding, spraying, vapor deposition, or the like.

Another alternative material for usage as the dissolvable substrate 102 is lecithin, which functions as a base or carrier for a deformable, solid structure with suitable cohesive and plastic characteristics. A lecithin compound may function either as a structural container for a whitening material 104 or may be mixed with the whitening material 104 to form an application unit.

Lecithin or a mixture of lecithin and whitening material 104 may be mixed, blended and kneaded into a viscous, homogeneous dough-like consistency, then shaped into a suitable mass, for example by extruding from a plastic mass form into one or more elongated rods. The formed shape may be sliced by a cutting edge into small tablet or capsule units of one or more selected shapes and sizes. Application units may be coated with one or more of a variety of tablet coating materials to control moisture penetration and stabilize appearance. Small quantities of additives such as granulating agents and disintegrants, for example cellulose and defatted soy polysaccharides, may be included with the dissolvable substrate 102 or mixture of whitening material 104 and dissolvable substrate 102. Furthermore, lecithin can be blended with relatively large amounts (2 to 4 times the weight of lecithin) of nearly any vitamin, mineral, or pharmaceutical substance, and retain suitable plastic mechanical characteristics. A small amount (for example 0.5% to 5% by weight) of oil, such as vegetable oil, may be added to lecithin to increase plasticity of the mixture. In some examples, the added oil may also include an active additive substance, for example vitamin E or vitamin A, further enhancing plasticity of the mixture.

In a further example, the dissolvable substrate 102 can be composed of a soft, deformable, dissolvable material such as a gummi candy material. Gummi candy is typically made, for example, from corn syrup, sugar, gelatin, and citric acid. Artificial sweeteners such as aspartame, saccharin, sucralose, xylatol, and the like may otherwise be used to flavor the dissolvable substrate 102.

Thickeners may be used in the dissolvable substrate 102 to reduce the dissolution rate and increase contact time of the whitening material 104. Suitable thickeners include neutralized carboxypolymethylene and other polyacrylic acid polymers and copolymers, hydroxypropylcellulose and other cellulose ethers, salts of poly(methyl vinyl ether-co-maleic anhydride), poly(vinylpyrrolidone), poly(vinylpyrrolidone-co-vinyl acetate), silicon dioxide, fumed silica, stearic acid esters, and others. A suitable concentration of thickener depends upon thickener type, but generally has a range up to about 20.0 percent by weight of the composition.

Artificial colorings may be added to the dissolvable substrate 102 composition to produce a desirable product color. Sweeteners or flavorings may be added to the dissolvable substrate 102 composition to enhance product desirability. Suitable sweeteners may include compressible confectioner's sugar, fructose, sorbitol, mannitol, xylitol, cyclamates, acesulfame K, thaumatin, sucralose, alitame, PS99/PS100, glycyrrhizin, monellin, stevioside, miraculin, L-sugars, or asparatame. The selected sweetener or combination of sweeteners and flavorings should be compatible with the whitening material 104 and the other components. Maltodextrin and cyclodextran may also be added to improve product taste by dissipating unpleasant flavors.

A flavor enhancer such as ribotide (a nucleotide) and monosodium glutamate (“msg”) may be included in the dissolvable substrate 102 composition.

A buffering material may be added to the dissolvable substrate 102 composition to produce a more palatable product with a whitening material 104 that is either severely acidic and sour, or severely basic and bitter. A suitable buffer system such as citric acid/sodium citrate or phosphate buffer system may be desirable for inclusion into the dissolvable matrix.

For the various type of dissolvable substrate 102 that is selected, constituent ingredients are mixed and the mixture is shaped into a desired solid form by a conventional technique, such as, for example, that including either extruding, molding, or rolling. The shaped form is then cut into a plurality of application units ready for packaging and distribution. The application units may be various shapes including tooth-shaped, rectangular, square, round, triangular, or any other suitable shape for application to one or more teeth. The units may be individual or connected, for example at a perforation, crease, or fold, so that individual units may be torn apart for application to a single tooth, or left connected for application to multiple teeth.

Application units may be uncoated or coated to give a finished appearance and resistance to moisture.

The whitening material 104 is a material capable of whitening teeth, generally by bleaching or polishing action, or both, that can be held within reservoirs or vesicles inside the structure of the dissolvable substrate 102, or can be mixed with the dissolvable substrate 102. The whitening material 104 acts on a user's teeth when the dissolvable substrate 102 is attached to the teeth during slow dissolution.

A whitening material 104 may be any suitable whitening material or compound, such as a peroxide compound, typically carbamide peroxide or hydrogen peroxide, or a bleaching agent.

The whitening material 104 may a liquid, gel, or paste that whitens stained or discolored teeth by a chemical process operating while in contact with the surface of the teeth for a period determined by the rate of dissolution of the dissolvable substrate 102.

For some whitening materials 104, the tooth-whitening compositions act by oxidation. The oxidizing compositions may contain a hydrogen peroxide precursor, for instance, carbamide peroxide, which may be mixed with an anhydrous or low water content, hygroscopic viscous carrier containing glycerin and/or propylene glycol and/or polyethylene glycol.

Other suitable whitening materials 104 include chlorine dioxide. The chlorine dioxide compound may not be stable for extended periods of time. In some examples, including two separate precursor materials for whitening as shown in FIG. 2 extends stability of the apparatus. The whitening material 104 is apportioned into two separate formulated portions including a first precursor 202 and a second precursor 204. In one example, one portion includes a chloride dioxide precursor such as sodium chlorite, and the second portion contains an acidulant. The chloride dioxide precursor generally has a pH greater than 7 to prevent premature generation of chloride dioxide during storage. The acidulant is formulated so that chloride dioxide is released when the acidulant combines with or contacts the chloride dioxide precursor and the resulting composition or interface has a pH of less than 7. A suitable embodiment includes a gelled aqueous chloride dioxide precursor portion containing 5000 parts per million of sodium chlorite, and a gelled acidulant portion containing 2.0% anhydrous citric acid. The composition formed by combination of the two portions may be placed in contact with a stained or discolored tooth surface to whiten the tooth. The two portions may be separated by a material such as the dissolvable substrate 102 material to avoid premature combination.

For example, the teeth whitening apparatus 200 may be utilized by compressing onto a tooth with the compression causing the admixture of the first precursor 202 and the second precursor 204 to initiate whitening action.

One suitable whitening material 104 is a peroxide compound that acts by bleaching and/or cleansing of tooth surfaces. Suitable peroxide compounds include metal peroxides such as calcium peroxide, magnesium peroxide, barium, peroxide, and zinc peroxide. Another suitable peroxide is carbamide peroxide, a hydrogen peroxide precursor that is typically mixed with an anhydrous or low-water content, hygroscopic viscous carrier containing glycerin and/or propylene glycol and/or polyethylene glycol. Any suitable concentration of peroxide compound may be utilized and the concentration may vary according to the particular dissolvable substrate 102 and the particular whitening material 104 that is employed. In one example, a suitable concentration of peroxide in the teeth whitening apparatus 100 ranges from about 0.25% to approximately 5% by weight.

The whitening material 104 may utilize any suitable peroxide compound of the many available peroxides including, most commonly, hydrogen peroxide and adducts or association complexes such as carbamide peroxide and sodium percarbonate. Hydrogen peroxide may remove discoloration by oxidizing unsaturated carbon-carbon, carbon-oxygen, and carbon-nitrogen bonds found in the stain molecules. Related compounds called peroxyacids remove stains by specific binding to certain stain molecules. Suitable whitening materials may include stable, solid peroxyacids including diperoxydodecanoic acid and the magnesium salt of monoperoxyphthalic acid. Other peroxyacids, such as peroxyacetic acid, may be used such as solutions containing an equilibrium distribution of acetic acid, hydrogen peroxide, peroxyacetic acid and water. Other suitable peroxide compounds may include a peroxide donor such as sodium perborate or sodium percarbonate in combination with a peroxyacid precursor. Examples of peroxyacids include peroxyacetic acid from hydrogen peroxide and tetraacetylethylenediamine, and peroxynonanoic acid from hydrogen peroxide and nonanoyloxybenzene sulfonate.

Utilization of a peroxyacid precursor with the dissolvable substrate 102 may improve whitening performance by prolonging and controlling the release of peroxyacid onto the teeth.

In some examples, a peroxide compound may be included in combination with an alkaline abrasive agent such as a bicarbonate compound, for example sodium bicarbonate. Other examples of suitable alkaline abrasive agents may include alkali metal compounds such as alkali metal hydroxides, carbonates, sesquicarbonates, borates and silicates specific examples of which are sodium hydroxide, potassium hydroxide, sodium carbonate, potassium carbonate, sodium borate, sodium sesquicarbonate and sodium silicate. Any suitable concentration of bicarbonate compound may be used and the concentration may vary according to the particular dissolvable substrate 102 and the particular whitening material 104 that is used. In one example, a suitable concentration of bicarbonate compound in the teeth whitening apparatus 100 ranges from about 5% to approximately 20% by weight. Particle size of the bicarbonate compound typically ranges from about 10 microns to about 300 microns.

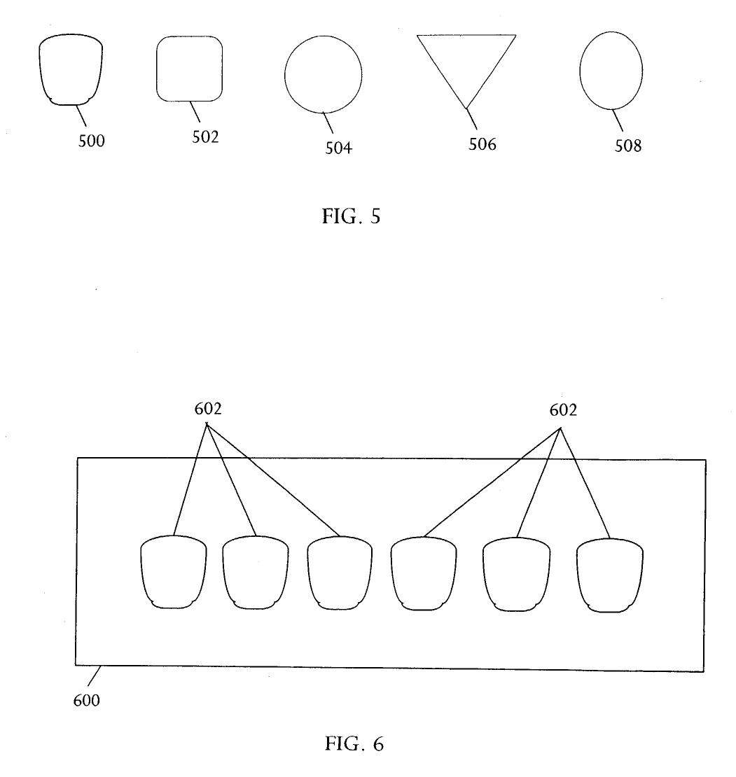

To increase the stability of a peroxide-bicarbonate combination, the peroxide and bicarbonate materials may be separated until use. In one example, as shown in FIG. 4 an application unit may be in the form of a dissolvable substrate 102 with interspersed reservoirs or vesicles, one or more of which contain a peroxide compound, and one or more of which contain a bicarbonate compound. The peroxide compound and bicarbonate compound, if placed in contact would be reactive. Accordingly, the peroxide compound is contained in one or more reservoirs or vesicles while the bicarbonate compound is contained in one or more other reservoirs or vesicles so that the reactive materials are separated. A user employs the application unit by pressing or crushing the dissolvable substrate 102 against one or more teeth, adhering the application to the teeth and combining the peroxide and bicarbonate compounds, thereby mixing the reactive peroxide and bicarbonate compounds in the aqueous environment, activating the whitening action of the combination.

In another example, the dissolvable substrate 102 may include one or more vesicles or reservoirs that contain a compound that includes both peroxide and carbonate, and further includes a humectant such as polyethylene glycol to improve storage stability of the reactive ingredients. A suitable concentration of polyethylene glycol is approximately 1% to 5% by weight.

In some applications a surfactant may be included in the whitening material 104 to aid in prophylactic action and to thoroughly disperse the whitener through the oral cavity. In other applications, no surfactant may be used so that the whitening material 104 remains localized to a particular tooth. Suitable organic surfactants include salts of higher alkyl sulfates, such as sodium lauryl sulfate (SLS) or other suitable alkyl sulfate, salts of sulfonated monoglycerides of higher fatty acids, such as sodium coconut monoglyceride sulfonate or other suitable sulfonated monoglycerides of a fatty acids. Other suitable organic surfactants may include salts of amides of higher fatty acid with lower aliphatic amino acids, such as sodium-N-methyl-N-palmitoyi tauride, sodium N-lauroyl-, N-myristoyl- and N-palmitoyl sarcosinates. Still other surfactants include salts of esters of fatty acids with isothionic acid or with glycerol monosulfate, such as the sodium salt of monosulfated monoglyceride of hydrogenated coconut oil fatty acids. Other surfactants are salts of olefin sulfonates, for example alkene sulfonates or hydroxalkene sulfonates. Suitable concentrations of surfactants range from about 0.5 to about 3.0% by weight.

In some examples, the whitening material 104 may include polishing agents. Other examples omit polishing agents. A suitable class of polishing agents includes siliceous materials, such as silica. Other suitable polishing agents are peroxide reactive polishing agents such as sodium bicarbonate, calcium carbonate, sodium metaphosphate, potassium metaphosphate, tricalcium phosphate, calcium phosphate dihydrate, anhydrous dicalcium phosphate, calcium pyrophosphate, magnesium orthophosphate, trimagnesium phosphate, alumina trihydrate, aluminum silicate, zirconium silicate, calcined alumina and bentonite. A suitable concentration for a polishing agent varies according to the particular agent and the particular whitening material 104. In some cases a suitable concentration of polishing agent ranges from about 5% to approximately 25% by weight of the whitening compound.

In some applications, the whitening material 104 may include inorganic thickeners. Other applications omit inorganic thickeners. Suitable inorganic thickeners include fumed silicas, thickening silicas, natural and synthetic gums and colloids. Particular examples of thickeners include carrageenan, xanthan gum and sodium carboxymethyl cellulose, starch, polyvinylpyrrolidone, hydroxyethylpropylcellulose, hydroxybutyl methyl cellulose, hydroxypropyl methyl cellulose, and hydroxyethyl cellulose. Suitable concentrations of inorganic or organic thickeners range from about 0.05 to about 2% by weight. In some applications, the whitening material 104 may include fluoride salts. In other applications, fluoride salts are omitted. Fluorine-providing salts are sometimes considered to have anti-caries efficacy Fluoride salts are water-soluble inorganic metal salts, for example, sodium fluoride, potassium fluoride, sodium monofluorophosphate and sodium fluorosilicate.

Some applications include colorants in the whitening material 104. Other applications omit colorants. Colorants include pigments and dyes. Pigments include non-toxic, water insoluble inorganic pigments such as titanium dioxide and chromium oxide greens, ultramarine blues and pinks and ferric oxides. A suitable range of pigment concentrations is from 0.5% to 3% by weight. Dyes are generally food color additives presently certified under the Food Drug & Cosmetic Act for use in food and ingested drugs.

The whitening material 104 may include or omit flavorings or sweeteners. Examples of suitable flavoring constituents are flavoring oils, such as oils of spearmint, peppermint, wintergreen, anethole, menthol, vanilla, sassafras, clove, sage, eucalyptus, marjoram, cinnamon, lemon, orange, other citrus flavors, and methyl salicylate. Suitable sweetening agents include sucrose, lactose, maltose, sorbitol, sodium cyclamate, perillartine, and sodium saccharin.

Various other materials may be incorporated into the whitening material 104 or omitted. Such materials may include preservatives, silicones and chlorophyll compounds, antibacterial agents such as chlorohexidene, halogenated diphenyl ethers such as Triclosan, desensitizing agents such as potassium nitrate and potassium citrate.

One particular example of a peroxide tooth-bleaching composition for contacting a tooth surface includes a hydrogen peroxide-containing compound, and a matrix for administering the hydrogen peroxide-containing compound to the tooth surface. In one example, the matrix includes a thickening agent, an agent for stabilizing the hydrogen peroxide-containing compound, a pH adjusting agent, and a calcium chelating agent. The bleaching reaction in a tooth using a chemical tooth-bleaching agent such as hydrogen peroxide may be enhanced at a pH greater than 5.5 with buffers included to maintain a relatively constant pH level. Usage of a calcium chelating agent avoids precipitation of calcium ions. Suitable pH adjusting agents include, but are not limited to, sodium hydroxide, potassium hydroxide, ammonium hydroxide, sodium carbonate, potassium carbonate, TRIS and triethanolamine. Suitable calcium chelating agents include EDTA and EDTA salts, citric acid and its salts, gluconic acid and its salts, alkali metal pyrophosphates and alkali metal polyphosphates.

In an alternative compound for usage as the whitening material 104, low concentrations of chlorine dioxide, for example in a range from 1 to 500 parts per million of total composition weight, may be contained within or released by a tooth whitening composition. The chlorine dioxide in combination with the tooth whitening composition, rapidly oxidizes stains when placed in contact with the tooth surface. In some applications, because chlorine dioxide may not be stable for extended time periods, the chlorine dioxide compound may be stored as separate precursors. For example, the precursors may be stored in different vesicles or reservoirs in the dissolvable substrate 102 until the teeth whitening apparatus 100 is pressed against a tooth during usage. For example, one portion may contain a chlorine dioxide precursor such as sodium chlorite, and another portion contains an acidulant. The chlorine dioxide precursor portion may generally be formulated to have a pH greater than 7 to prevent premature generation of chlorine dioxide during storage. The acidulant portion, is generally formulated so upon combination chlorine dioxide precursor, chlorine dioxide is released from the precursor and the resulting composition or interface has a pH of less than 7. In one example the tooth whitening composition includes a gelled aqueous CDP portion containing 5000 ppm sodium chlorite, and a gelled acidulant portion containing 2.0% anhydrous citric acid. Typical chlorine dioxide precursors may be alkali metal chlorites or alkali metal chlorates, such as sodium chlorite.

Depending upon the application, the chlorine dioxide precursor portion may contain a variety of auxiliary components for the purpose of stabilizing, thickening, or otherwise improving its performance in whitening teeth. Stabilizers for the chlorine dioxide precursor portion include, but are not limited to, alkali metal chlorides and alkali metal carbonates. Thickeners include, but are not limited to, hydroxyethylcellulose, polyethylene glycol and polyoxyethylene. Performance enhancers include, but are not limited to, non-ionic surfactants such as poly(cthylene oxide-co-propylene oxide) block copolymers.

The acidulant portion generally contains an aqueous carrier and a water-soluble acidulant. Suitable water-soluble acidulants may be citric acid, malic acid, fumaric acid, and other non-toxic, orally acceptable acidulants. A water-soluble acidulant may be a polymeric compound, such as carboxypolymethylene. The acidulant portion may also include auxiliary components including thickeners, performance enhancers, and preservatives. Suitable thickeners include partially neutralized carboxypolymethylene, polyoxyethylene, xanthan gum, and other acid-stable polymers.

Other examples of a suitable whitener include compositions that generate peroxyacetic acid upon contact with an aqueous medium or environment. The peroxyacetic acid functions as an oxidant in a tooth-whitening or stain-removal process. Various compounds are capable of generating peroxyacetic acids quickly and effectively for dental application.

Typically compounds capable of generating peroxyacetic acid include at least one orally acceptable acyl group source or precursor and at least one orally acceptable peroxide source. A peroxyacetic acid results from the interaction of the acyl group source and the peroxide precursor in an aqueous solution. In some applications, the dissolvable substrate 102 functions as an anhydrous carrier that separates the acyl group source from the peroxide precursor prior to application to the teeth.

In one example, the acyl group source is an acetyl group source with a low molecular weight, at least one acetyl group to be used in the formation of a peroxy acid, and a steric configuration that allows the acetyl group source to penetrate pores in teeth. When the acetyl group source has penetrated a tooth, a peroxide source can generate a peroxyacid within a tooth.

In a specific example, the acetyl group source is a low molecular weight C₁-C₅ molecule with between one and five labile acetyl groups. Any labile functional groups having similar properties to acetyl groups are suitable that form an agent useful in tooth whitening and stain removal.

Another example of a dissolvable substrate 102 that generates peroxyacetic acid is a composition that has at least two components including one component with a peroxide source such as hydrogen peroxide, and a second component including a source of acetyl groups. Typically, the two components may be mixed at the time of application, for example by crushing the dissolvable substrate 102 containing separate vesicles for the two components against one or more teeth. Alternatively, each component may be sequentially applied directly to the tooth surface. For example, a dissolvable substrate 102 containing a source of acetyl groups may be applied to the tooth and allowed to penetrate into pores present in the tooth structure for a sufficient time period while the dissolvable substrate 102 dissolves. Then the second component containing the peroxide precursor may be applied to generate peroxyacid deep within the tooth structure to interact with chromogens within the tooth structure to result in enhanced tooth whitening.

Suitable hydrogen peroxide precursors include but are not limited to carbamide peroxide, sodium percarbonate, sodium perborate, calcium peroxide, magnesium peroxide, sodium peroxide, and the anhydrous poly(vinyl pyrrolidone)/hydrogen peroxide complexes. Other suitable hydrogen peroxide precursors include any compounds that can generate, convert, or otherwise form hydrogen peroxide or peroxide anion when in contact with water. For example, alkali metal percarbonates such as potassium percarbonate, as well as enzymatic sources of hydrogen peroxide, such as glucose oxidase in combination with beta-D-glucose may be suitable in some applications.

A suitable peroxide precursor encased or mixed within the dissolvable substrate 102 may be applied directly to the tooth surface in an amount sufficient to result in a hydrogen peroxide concentration of from about 0.1 percent by weight to about 15 percent by weight.

Compositions that utilize hydrogen peroxide, rather than a precursor, are typically prepared as two or more components separated by the dissolvable substrate 102. The acetyl group source is contained in one component and hydrogen peroxide in a second component. A suitable source of labile acetyl groups is glyceryl triacetate, glyceryl diacetate or glyceryl acetate, in an amount sufficient to allow rapid generation of peroxyacid, for example in a range between about 0.1 percent by weight to about 6.0 percent by weight of the composition. Suitable carriers for single component compositions are toxicologically benign and may include glycerin, propylene glycol, and polyethylene glycols. Oil-based carriers may be used, for example in combination with a surfactant capable of emulsifying the composition upon contact with water. Suitable oils include vegetable and mineral oils, or waxes and esters. Carriers for multi-component compositions include all single component carriers and also water. Carriers may also include thickeners, buffering compounds, chelating agents, stabilizers, surfactants, sweeteners, and flavorants.

A buffer may be included to create a suitable pH for penetration of the composition into tooth enamel or to facilitate peroxyacetic acid generation from the hydrogen peroxide precursor and glyceryl triacetate. Suitable buffers include sodium hydroxide, potassium hydroxide, ammonium hydroxide, sodium phosphate di- and tri-basic, potassium phosphate di- and tri-basic, sodium tripolyphosphate, tris (hydroxymethyl) aminomethane, triethanolamine, polyethylenimine, and other alkaline buffers. An alkaline buffer may function to neutralize carboxylic acid side chains in thickening polymers such as polyacrylic acid and poly(methyl vinyl ether-co-maleic anhydride). Acid buffers such as citric acid, phosphoric acid, and others may also be used alone or in combination with an alkaline buffer to select pH and improve buffering capacity. A suitable concentration of a buffer ranges up to about 3.0 percent by weight of the composition.

Compositions may optionally contain one or more chelating agents to bind with metal ions in the composition that would otherwise degrade the hydrogen peroxide. Suitable chelating agents include phosphonic acids, EDTA, and polyphosphates and may be applied in a concentration of up to about 5.0 percent by weight of the composition.

Surfactants may be used to lower the surface tension of the compositions, allowing better wetting and spreading of the composition on the tooth surface. Some surfactants, for example zwitterionic and fluorinated surfactants, may increase penetration of compositions into the tooth structure. Suitable surfactant concentrations range up to about 2.0 percent by weight of the composition.

Another example of a suitable whitening material 104 is a smooth flowing thixotropic, high sheen (glossy), relatively water-free multiphase gel or paste formulation that contains whitener, an essentially anhydrous (free of bulk water) organic hydroxylated liquid matrix material, and a small quantity of a high molecular weight acidic polymer. The matrix material wets each component and allows the formation of the gelled state. Typically the matrix material may include one or more aliphatic organic polyols, for example glycerine and propylene glycols. The polymer may be a carboxylated polymer and/or silica having capacity to hydrogen bond with the aliphatic organic polyols to create a gel structure. In one suitable application, a stable homogenized essentially-anhydrous gel/paste is derived from granulated, finely and uniformly dispersed, esterified amylopectin and/or esterified amylose with a relatively small amount of a powdered carbomer polymer.

The thixotropic composition may include any suitable whitener such as hydrogen peroxide through carbamide peroxide, calcium peroxide, or other peroxide compounds. The thixotropic character of the whitening material 104 facilitates distribution of the bleaching agent on dental surfaces and aids adherence to the teeth, allowing the peroxide to become separated via enzymatic action and then decomposed by enzymatic action to form active oxidizing oxygen that whitens and treats gum surfaces. Ingredients in the thixotropic whitening material 104 may include organic polyol, such as glycerine and propylene glycol, the acylated amylopectin, the gelling agent, flavoring, and any active dental ingredient. Proportions of the components may vary, but generally the organic polyol comprises from about 20 weight percent to about 65 weight percent of the total weight. The amylopectin and/or the acylated amylopectin, generally alone or in admixture with amylose and/or acylated amylose, comprises from about 5 to about 35 weight percent of the weight. Other ingredients may include active ingredients, abrasives, surfactant, fillers, enzymes, stabilizers, and preservatives.

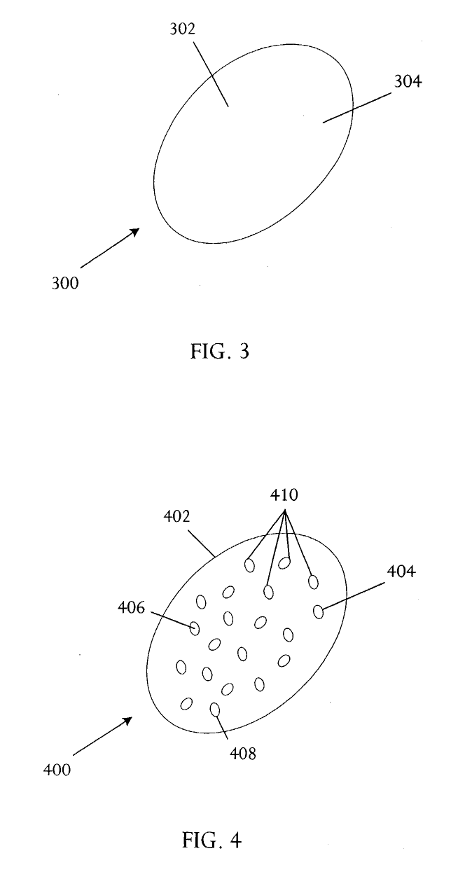

Referring to FIG. 3, a pictorial drawing shows an example of a teeth whitening apparatus 300 that includes a whitening material mixed into a dissolvable matrix. In some embodiments, the dissolvable substrate 102 functions as a structural shell for either partly or fully enclosing a reservoir capable of containing the whitening material 104. In other embodiments, the whitening material 304 may be mixed with the dissolvable substrate 302 to form a flexible structure capable of adhering to a dental surface. Whether the whitening material is mixed with or encased by the dissolvable substrate depends on compatibility and structural characteristics of the particular selected dissolvable substrate and whitening material.

Referring to FIG. 4, a pictorial drawing illustrates an example of a teeth whitening system including precursor materials 404, 406, and 408 distributed in vesicles 410 or reservoirs throughout a dissolvable matrix 402.

In the example, the dissolvable substrate 402 may be in the form of a gelatin capsule may include multiple pockets 410, with individual pockets containing precursor materials that combine when applied to a tooth to form an active whitening compound.

Referring to FIG. 5, several examples of suitable shapes of the teeth whitening apparatus 100 are shown. Suitable shapes in two dimensions include a tooth-shaped matrix 500, a square matrix 502, a circular matrix 504, a triangular shaped matrix 506, an oval matrix 508, or any other suitable shape. In a third dimension, the shapes may be any suitable forms such as a sheet of any suitable thickness, a curved sheet, or any regular or irregular form. In other examples, the shapes may have any suitable form, including but not limited to spherical, oblate, ellipsoidal, ovoid, and cylindrical shapes. The teeth whitening apparatus 100 may be any size and may cover a single tooth or multiple teeth. The teeth whitening apparatus 100 may be configured to fit an outer tooth surface, an inner tooth surface, or any other tooth surface.

The teeth whitening apparatus 100 elements may be configured in different sizes for the different teeth and for use on the inner tooth surface or outer tooth surface.

FIG. 6 shows an example of a suitable packaging configuration for the teeth whitening apparatus including a sterile mounting strip 600 upon which a plurality of teeth whitening elements 602 are mounted. In the illustrative embodiment, the plurality of teeth whitening elements 602 contact a mounting strip 600 such as a parafin-coated strip to facilitate removal. A user can peal an individual whitening element 602 from the strip 600 and compress the element 602 onto a tooth to affix the element to the tooth.

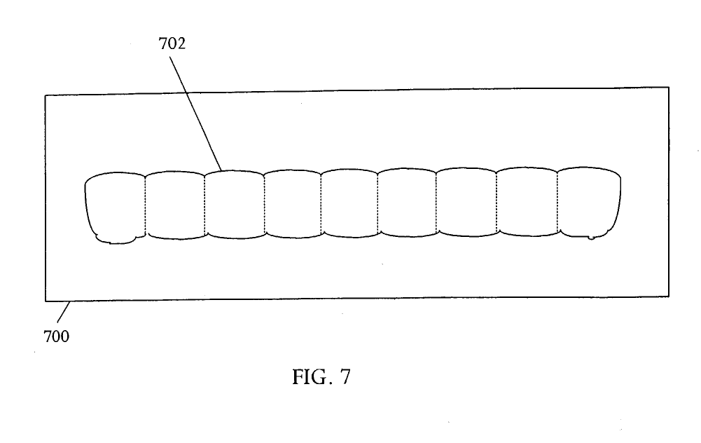

The mounting strip 600 is generally packaged in a sealed, sterile packing material. The whitening elements 602 may be attached individually as shown in FIG. 6, or may be connected in a strip 702 as shown in FIG. 7. The whitening elements 702 may have perforations, creases, folds, or the like to facilitate separation into individual elements.

The strip 702 includes elements that may be sectioned for usage on individual teeth or may be used in combination with several elements for application to several teeth. The elements may be trimmed for customization to a particular tooth or application.

While the invention has been described with reference to various embodiments, it will be understood that these embodiments are illustrative and that the scope of the invention is not limited to them. Many variations, modifications, additions and improvements of the embodiments described are possible. For example, those skilled in the art will readily implement the steps necessary to provide the structures and methods disclosed herein, and will understand that the process parameters, materials, and dimensions are given by way of example only and can be varied to achieve the desired structure as well as modifications which are within the scope of the invention. Variations and modifications of the embodiments disclosed herein may be made based on the description set forth herein, without departing from the scope and spirit of the invention as set forth in the following claims.

In the claims, unless otherwise indicated the article “a” is to refer to “one or an one”.

What is claimed is:

1. A teeth whitener comprising:
a dissolvable matrix that is capable adhering to a dental surface and dissolving in the presence of oral fluids over a time period of minutes or hours; and
a whitening material supported by the dissolvable matrix.

2. A teeth whitener according to claim 1 wherein:
the dissolvable matrix is in the form of a cup and the whitening material is contained within the cup.

3. A teeth whitener according to claim 1 wherein:
the dissolvable matrix is interspersed with one or more vesicles and the whitening material is contained within the one or more vesicles.

4. A teeth whitener according to claim 1 wherein:
the whitening material is admixed within the dissolvable matrix.

5. A teeth whitener according to claim 1 wherein:
the whitening material is composed of two or more precursor materials that are contained within separate vesicles within the dissolvable matrix.

6. A teeth whitener according to claim 1 wherein:
the dissolvable substrate is in the form of a solid, flexible matrix capable of conforming to the shape of a tooth or several teeth.

7. A teeth whitener according to claim 1 wherein:
the dissolvable substrate is in the form of a solid, flexible matrix capable of conforming to the shape of a tooth or several teeth.

8. A teeth whitener according to claim 1 wherein:
the dissolvable substrate is in the form of a flexible matrix that includes perforations, creases, or folds so that portions may be separated for application to one or more teeth.

9. A teeth whitener according to claim 1 wherein:
the dissolvable substrate is composed of a gelatin material.

10. A teeth whitener according to claim 1 wherein:
the dissolvable substrate is composed of a gelatin material including vegetable oil, gelatin, glycerin, and water.

11. A teeth whitener according to claim 1 wherein:
the dissolvable substrate is composed of a cellulose and acrylic polymer.

12. A teeth whitener according to claim 1 wherein:
the dissolvable substrate is composed of a matrix of materials selected from among carbohydrates, fats, proteins, waxes, dissolvable resins, and hydrogels..

13. A teeth whitener according to claim 1 wherein:
the dissolvable substrate comprises a lecithin compound.

14. A teeth whitener according to claim 1 wherein:
the dissolvable substrate is composed of a gummi candy material.

15. A teeth whitener according to claim 1 wherein:
the dissolvable substrate is composed of a gummi candy material including a sweetener, gelatin, and citric acid.

16. A teeth whitener according to claim 1 wherein:
the dissolvable substrate includes a hydrophobic agent that controls dissolution rate.

17. A teeth whitener according to claim 1 wherein:
the dissolvable substrate includes a hydrophobic agent that controls dissolution rate selected from among calcium stearate to slow dissolution and lactose to enhance dissolution.

18. A teeth whitener according to claim 1 wherein:
the whitening material comprises a peroxide bleaching agent.

19. A teeth whitener according to claim 1 wherein:
the whitening material comprises a peroxide bleaching agent selected from among hydrogen peroxide, carbamide peroxide, calcium peroxide, magnesium peroxide, barium peroxide, zinc peroxide, hydrogen peroxide association complexes of carbamide peroxide and sodium percarbonate, and peroxyacids.

20. A teeth whitener according to claim 1 wherein:
the whitening material comprises a peroxide bleaching agent in combination with alkaline abrasive agents selected from among alkali metal hydroxides, alkali metal carbonates, alkali metal sesquicarbonates, alkali metal borates, alkali metal silicates, sodium hydroxide, potassium hydroxide, sodium carbonate, potassium carbonate, sodium borate, sodium sesquicarbonate, and sodium silicate.

21. A teeth whitener according to claim 1 wherein:
the whitening material comprises chloride dioxide.

22. A teeth whitener according to claim 1 wherein:
the whitening material comprises chloride dioxide apportioned in separate precursor materials including a chloride dioxide precursor and an acidulent.

23. A teeth whitener according to claim 1 wherein:
the whitening material comprises a thixotropic formulation with a whitener, an essentially anhydrous organic hydroxylated liquid matrix material, a high molecular weight acidic polymer.

24. A teeth whitener according to claim 1 further comprising:
a thickening agent that reduces dissolution rate.

25. A teeth whitener according to claim 1 further comprising:
one or more flavoring material.

26. A teeth whitener according to claim 1 further comprising:
one or more buffering materials.

27. A teeth whitener according to claim 1 further comprising:
one or more surfactants.

28. A teeth whitener according to claim 1 further comprising:
one or more polishing agents.

29. A teeth whitener according to claim 1 further comprising:
one or more chelating agents.

30. A teeth whitener according to claim 1 wherein:
the dissolvable substrate is formed into a shape selected from among tooth-shaped, square, circular, triangular, oval, spherical, oblate, ellipsoidal, ovoid, and cylindrical.

31. A teeth whitening system comprising:
a sterile strip;
a dissolvable matrix mounted on the sterile strip that is capable of dissolution in the presence of oral fluids; and
a whitening material contained by the dissolvable matrix.

32. A teeth whitening system according to claim 31 wherein:
the whitening material is contained between the sterile strip and the dissolvable matrix.

33. A teeth whitening system according to claim 31 wherein:
the whitening material is contained in vesicles within the dissolvable matrix.

34. A teeth whitening system according to claim 31 wherein:
the whitening material is mixed within the dissolvable matrix.

35. A teeth whitening product comprising:
a dissolvable substrate that is capable of adhering to a tooth surface and gradually dissolving from the tooth surface in a time period that ranges up to one or more hours; and
a whitening material contained by the dissolvable matrix that is released during dissolution of the dissolvable substrate.

36. A method of supplying a teeth whitening product comprising:
fabricating a dissolvable substrate that is capable of adhering to a tooth surface and gradually dissolving from the tooth surface in a time period that ranges up to one or more hours;
containing a whitening material within the dissolvable matrix; and
gradually releasing the whitening material as dissolvable substrate dissolves.
